(19) United States
(12) Patent Application Publication
(10) Pub. No.: US 2024/0000653 A1
(43) Pub. Date: Jan. 4, 2024

(54) THERMOTHERAPY DEVICE

(71) Applicant: CERAGEM CO., LTD., Cheonan-si, Chungcheongnam-do (KR)

(72) Inventors: Dong Myoung Lee, Asan-si, Chungcheongnam-do (KR); Keun Young Paek, Cheonan-si, Chungcheongnam-do (KR); Sang Ho Choi, Cheonan-si, Chungcheongnam-do (KR)

(21) Appl. No.: 18/254,750

(22) PCT Filed: Nov. 24, 2021
(86) PCT No.: PCT/KR2021/017413

§ 371 Date: May 26, 2023

(30) Foreign Application Priority Data
Nov. 30, 2020 (KR) 10-2020-0163876

(51) Int. Cl. A61H 15/02

(52) U.S. Cl. CPC A61H 15/02; A61H 2203/0443; A61H 2201/0207

(57) ABSTRACT

A thermotherapy device is capable of being supplied with an electric current from a power supply part in a non-rotating state even when a heating part for heating a ceramic part rotates together with the ceramic part. To this end, the thermotherapy device comprises a ceramic part having an inner space formed therein; a heating part which includes a heating element inserted into the inner space and emitting heat such that the ceramic part is heated, and a transfer member for transferring, to the ceramic part, heat generated from the heating element; a power supply part having an electrode member so as to supply an electric current to the heating part; and a support part for supporting the ceramic part, wherein the heating part may rotate relative to the electrode member such that the heating part rotates together with the ceramic part.

### TECHNICAL FIELD

The present invention relates to a thermotherapy device. More specifically, it relates to a thermotherapy device which is capable of receiving a current from a power supply part even in a state where the heating part for heating a ceramic part rotates together with the ceramic part.

### BACKGROUND ART

Conventionally, thermotherapy devices that relieve acute or chronic pain occurring in the muscles and nervous tissue of the spine caused by continuing work for a long period of time in an inappropriate posture or habituating this posture for a long period of time, move along the body part to improve the blood circulation of the body or relieve momentary muscle stiffness, and improve blood circulation through stimulation by heating in the area where pain occurs have been widely used.

The conventional thermotherapy device used for such thermotherapy performs massage while a heating ceramic is moved in the longitudinal direction along the user's body, and the heating ceramic is configured to rotate in the process of repeatedly reciprocating the entire moving section to massage the user's body. This is to configure a heating ceramic to rotate naturally due to the friction of a cover, because if the heating ceramic does not rotate, the friction between the heating ceramic and the cover may be maximized, and the cover may be quickly worn out.

In the conventional case, a heating element in a non-rotating state which is connected to a power source is inserted into the heating ceramic in order to heat the rotating heating ceramic, and the heating ceramic is configured to be spaced apart from the heating element such that the rotating heating ceramic can rotate relative to the heating element in a non-rotating state.

However, as the heating ceramic and the heating element are disposed to be spaced apart from each other, the heat generated from the heating element is not smoothly transferred, and thus, there is a problem in that the thermotherapy effect is reduced.

Therefore, there is a need for improvement in these areas.
(Patent Document 1) Korean Registered Patent No. 2002-0039608 (published on May 27, 2002)

### DISCLOSURE

### Technical Tasks

The technical problem to be solved in the present invention is directed to solving the problems of the related art described above, and it is directed to providing a thermotherapy device which is capable of receiving a current from a power supply part even in a state where the heating part for heating a ceramic part rotates together with the ceramic part.

The technical problems to be solved in the present invention are not limited thereto, and other technical problems that are not mentioned will be clearly understood by those skilled in the art from the following description.

### Technical Solution

In order to solve the above-described technical problems, the thermotherapy device according to the present invention includes a ceramic part having an inner space formed therein; a heating part which is inserted in the inner space and has a heating element for generating heat so as to heat the ceramic part, and a transfer member for transferring the heat generated by the heating element to the ceramic part; a power supply part having an electrode member so as to supply an electric current to the heating part; and a support part for supporting the ceramic part, wherein the heating part rotates relative to the electrode member such that the heating part rotates together with the ceramic part.

In this case, the electrode member may include a head which electrically contacts the transfer member and supports the transfer member so as to be rotatable, and a body which is fixed to the support part to support the head.

In this case, the head may support the transfer member in a manner of rotating with the transfer member, and the body may support the head to rotate relatively.

In this case, the body may be provided with a spring for providing a pressing force such that the head presses the transfer member.

In this case, a conducting groove may be formed in the transfer member to be in surface contact with the head while surrounding a portion of the outer peripheral surface of the head.

In this case, the head may support the transfer member to relatively rotate in a fixed state in a non-rotating state, and the body may support the head.

In this case, a deformable surface which is elastically deformed to press the transfer member may be formed on the head.

In this case, the deformable surface may be elastically deformed in such a way that it protrudes toward the transfer member.

In this case, the transfer member may be provided with an electrode plate that electrically contacts the head.

In this case, a contact surface may be formed on the electrode plate to be in surface contact with the head while surrounding a portion of the outer peripheral surface of the head.

In this case, the heating part may be provided with an elastically deformable member that presses the inner peripheral surface of the ceramic part.

### Advantageous Effects

Since the thermotherapy device of the present invention having the above configuration is configured such that the heating part for heating the ceramic part rotates together with the ceramic part, the heat generated in the heating part is smoothly transferred to the ceramic part, as the ceramic part and the heating part are disposed to contact with each other, thereby improving the thermotherapy effect.

In addition, as heat is smoothly transferred from the heating part to the ceramic part, heat loss is minimized, and thus, the power consumption efficiency of the thermotherapy device is improved.

Moreover, since the current is stably supplied even in a state where the heating part rotating together with the ceramic part rotates relative to the power supply part, it is possible to secure the operation stability of the thermotherapy device.

It should be understood that the effects of the present invention are not limited to the above-described effects, and include all effects that can be inferred from the configuration of the invention described in the detailed description or claims of the present invention.

### DESCRIPTION OF DRAWINGS

FIG. 1 is a cross-sectional view showing a thermotherapy device according to an exemplary embodiment of the present invention.

FIG. 2 is a cross-sectional view illustrating a state in which a ceramic part and a heating part are coupled according to an exemplary embodiment of the present invention.

FIG. 3 is a cross-sectional view illustrating an electrode member according to an exemplary embodiment of the present invention.

FIG. 4 is a cross-sectional view illustrating a heating part according to another exemplary embodiment of the present invention.

FIG. 5 is a cross-sectional view illustrating an electrode member according to another exemplary embodiment of the present invention.

FIG. 6 is a cross-sectional view illustrating a state in which the ceramic part and the heating part are coupled according to still another exemplary embodiment of the present invention.

FIG. 7 is a cross-sectional view illustrating an electrode plate and an electrode member coupled to each other according to various exemplary embodiments of the present invention.

FIG. 8 is a perspective view illustrating a heating part according to an exemplary embodiment of the present invention.

FIG. 9 is an exploded perspective view of a heating part according to an exemplary embodiment of the present invention.

FIG. 10 is a side view illustrating a heating part according to still another exemplary embodiment of the present invention.

FIGS. 11 to 14 are cross-sectional views illustrating an electrode member according to various exemplary embodiments of the present invention.

### MODES OF THE INVENTION

Hereinafter, with reference to the accompanying drawings, the exemplary embodiments of the present invention will be described in detail so that those of ordinary skill in the art can easily practice the present invention. The present invention may be embodied in many different forms and is not limited to the exemplary embodiments described herein. In order to clearly describe the present invention in the drawings, parts that are irrelevant to the description are omitted, and the same reference numerals are assigned to the same or similar components throughout the specification.

In the present specification, terms such as “include” or “have” are intended to designate that a feature, number, step, operation, component, part or combination thereof described in the specification exists, but it should be understood that it does not preclude the possibility of the presence or addition of one or more numbers, steps, operations, components, parts or combinations thereof. In addition, when a part of a layer, film, region, plate and the like is said to be “on” another part, this includes not only cases where the other part is “directly on”, but also cases where there is another part therebetween. Conversely, when a part of a layer, film, region, plate and the like is said to be “under” another part, this includes not only cases where it is “directly under” another part, but also cases where there is another part therebetween.

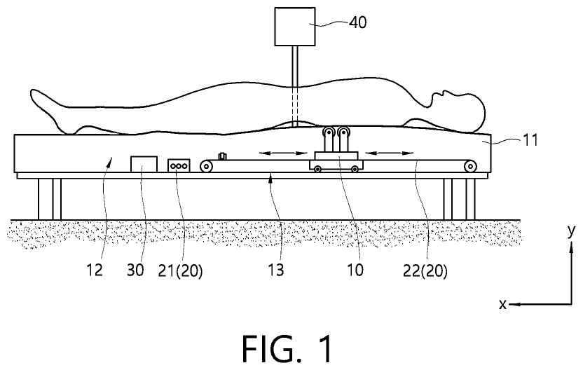

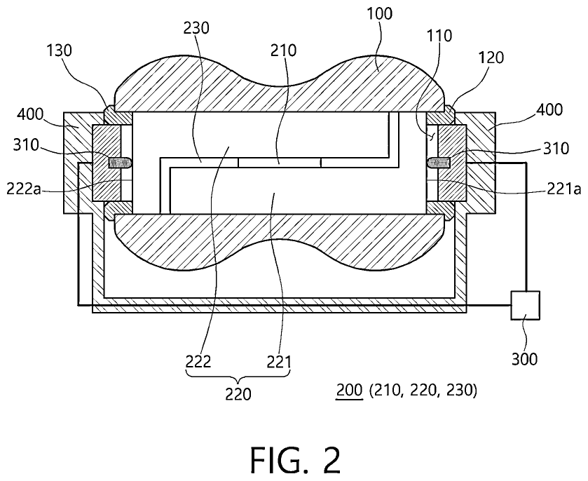

FIG. 1 is a cross-sectional view showing a thermotherapy device according to an exemplary embodiment of the present invention, and FIG. 2 is a cross-sectional view illustrating a state in which a ceramic part and a heating part are coupled according to an exemplary embodiment of the present invention.

As illustrated in FIG. 1, the thermotherapy device according to an exemplary embodiment of the present invention includes a ceramic module 10, a driving part 20 for moving the ceramic module 10, a control part 30 for controlling the operation of the driving part 20, and an input part 40 for inputting a desired thermotherapy pattern by the user.

In this case, such a thermotherapy device may include a main mat 11 which is used for the user's upper body and the spine portion thereof, and an auxiliary mat 12 which is used for the user's lower body part as a target. In addition, it may include a mounting part 13 for placing and supporting the main mat 11 and the auxiliary mat 12, as necessary.

The ceramic module 10 may massage the spine while moving in the longitudinal direction (x) along the user's spine, and the ceramic module 10 may provide a thermal compress and massage effect to the user by using high-temperature heat generated by using a current supplied from a power supply part 300 to be described below.

In this case, the ceramic module 10 may be configured to provide the user with a thermal compress and massage effect by using not only high-temperature heat but also far-infrared rays.

The ceramic part 100 provided in the ceramic module 10 may be formed in a roller type, but the present invention is not limited thereto, and if the ceramic part 100 is configured to rotate while the ceramic module 10 is moved, various shapes and structures are possible. In addition, when the ceramic part 100 is formed of a material such as ceramic, far-infrared rays are generated in the process of using the thermotherapy device, and thus, the thermotherapy effect may be improved. However, the present invention is not necessarily limited to these materials, and it may be formed of other materials as long as they can transfer heat to the user's body and provide a thermotherapy effect.

As illustrated in FIG. 2, the ceramic module 10 includes a ceramic part 100 having an inner space 110 formed therein, a heating part 200 which is inserted into the inner space 110 and has a heating element 210 for generating heat such that the ceramic part 100 is heated and a transfer member 220 for transferring the heat generated from the heating element 210 to the ceramic part 100, a power supply part 300 for supplying a current to the heating part 200, and a support part 400 for supporting the ceramic part 100.

Herein, a PTC heater may be used as the heating element 210, but the present invention is not limited thereto, and a lamp which is capable of heating by supplying a current or various heating elements may be used.

In addition, the driving part 20 may include a first driving member for moving the ceramic module 10 in the longitudinal direction (x) of the user. The first driving member may include a driving motor 21 and a transport member 22 for reciprocating the ceramic module 10.

The driving motor 21 receives a current to rotate, and the transport member 22 is connected to the driving motor 21 and transmits the rotational force according to the rotation of the driving motor 21 to move the ceramic module 10.

The transport member 22 is connected to the ceramic module 10, and according to the forward or reverse rotation of the driving motor 21, it is used to transfer the ceramic module 10 in one side direction or the other side direction along the longitudinal direction (x) of the user.

The transport member 22 may be selectively used among a transfer belt, a transfer chain and a transfer rope, but the present invention is not limited thereto, and various means for transporting an object by using the driving force of the driving motor 21 may be used, such as a lead screw or a method of using a rack and pinion.

The driving motor 21 may be configured to provide a driving force while being spaced apart from the magnetic module 10 or to provide a driving force while being inserted into the magnetic module 10.

Moreover, the driving part 20 may include a second driving member for raising the vertical height of the ceramic module 10 along the height direction (y) such that the pressing force is applied to the user or lowering the vertical height of the ceramic module 10 such that the pressing force is removed.

In this case, as illustrated in FIG. 2, in the thermotherapy process, the heating part 200 is configured to rotate together so as to rotate with the ceramic part 100, and thus, as the ceramic part 100 and the heating part 200 are disposed to be in contact with each other, the heat generated from the heating part 200 is smoothly transferred to the ceramic part 100, thereby improving the thermotherapy effect.

The heating part 200 may be configured to evenly contact the entire inner peripheral surface of the inner space 110 formed in the ceramic part 100, but the present invention is not necessarily limited thereto, and if the heat generated from the heating part 200 can be smoothly transferred to the ceramic part 100, it is also possible to configure the same to contact each other only at certain parts.

In this way, as heat is smoothly transferred from the heating part 200 to the ceramic part 100, heat loss is minimized and the power consumption efficiency of the thermotherapy device is improved, and the heating part 200 is stably supplied with a current through the power supply part 300 while rotating relative to the power supply part 300 such the thermotherapy device can operate stably. In this case, although relative rotation of the heating part 200 and the power supply part 300 may occur as the power supply part 300 is fixed in a non-rotating state, depending on some cases, the power supply part 300 also rotates at a constant speed, but relative rotation may occur between the heating part 200 and the power supply part 300 as it rotates slower than the rotational speed of the heating part 200 (low-speed rotation state).

The power supply part 300 may be provided with an electrode member 310 to be described below, and the electrode member 310 may be configured to smoothly supply a current even in a state where the heating part 200 rotates together with the ceramic part 100.

As illustrated in FIG. 2, a first bushing 120 is provided on one side of the ceramic part 100, and a second bushing 130 is provided on the other side of the ceramic part 100 such that the ceramic part 100 is rotatably supported by the support part 400.

That is, the heating part 200 is inserted into the inner space 110 of the ceramic part 100 in a state where the first bushing 120 is coupled to one side of the ceramic part 100. In this case, the heating part 200 may be inserted in a state where the heating element 210 and the transfer member 220 are assembled with each other, or sequentially inserted in a state where the heating element 210 and the transfer member 220 are separated.

As illustrated in FIG. 2, the transfer member 220 may include a first transfer body 221 and a second transfer body 222 that are disposed to face each other, and the above-described heating element 210 may be provided between the first transfer body 221 and the second transfer body 222.

That is, by disposing the first transfer body 221 to contact on one side surface of the heating element 210, and the second transfer body 222 to contact on the other side surface of the heating element 210, the heat generated by the heating element 210 may be transferred to the first transfer body 221 and the second transfer body 222, and the heat that has been transferred to the first transfer body 221 and the second transfer body 222 is transferred to the ceramic part 100. Transfer surfaces contacting the inner peripheral surface of the inner space 110 may be formed on the outer peripheral surface of the first transfer body 221 and the outer peripheral surface of the second transfer body 222, respectively.

As described above, if the heat generated through the heating element 210 is configured to be directly transferred to the ceramic part 100 in a conductive manner through the first transfer body 221 and the second transfer body 222, the heat transfer performance may be improved such that the thermotherapy effect is enhanced.

In this case, as illustrated in FIG. 2, the power supply part 300 may have an electrode member 310 which is disposed on one side of the ceramic part 100 and a second electrode 312 which is disposed on the other side of the ceramic part 100.

By configuring such that the current supplied through the power supply part 300 moves to the heating element 210 through the electrode member 310 on one side and the current passing through the heating element 210 moves back to the power supply part 300 through the electrode member 310 on the other side, it is possible to enable a smooth current supply.

The electrode members 310 are fixed to a non-rotating or low-speed rotation state through the support part 400, and they are in electrical contact with the heating part 200 to supply a current to the heating part 200 that rotates together with the ceramic part 100. That is, relative rotation occurs between the electrode member 310 and the heating part 200, and in this way, in order to stably supply a current even in the process of relative rotation, it is important that the electrical contact between the electrode member 310 and the heating part 200 is stably maintained.

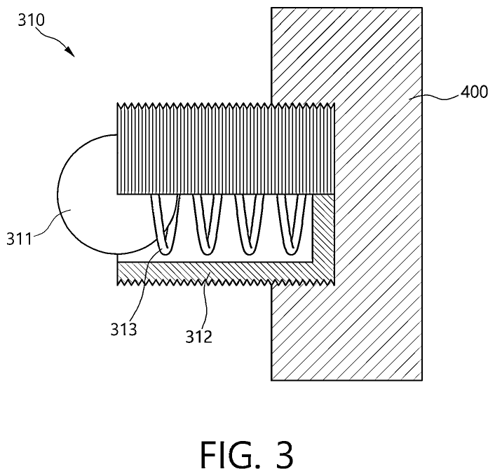

FIG. 3 is a cross-sectional view illustrating an electrode member according to an exemplary embodiment of the present invention.

As illustrated in FIG. 3, the electrode member 310 may include a head 311 for supporting the transfer member 220 to be rotatable while in electrical contact with the transfer member 220, and a body 312 which is fixed to the support part 400 to support the head 311.

That is, the first transfer body 221 provided in the transfer member 220 is in electrical contact with the head 311 of the electrode member 310 on one side, and the second transfer body 222 is in electrical contact with the head 311 of the electrode member 310 on the other side.

To this end, as illustrated in FIG. 2, a first conductive surface 221a which is in electrical contact with the electrode member 310 may be formed on the first transfer body 221, and a second conductive surface 222a which is in electrical contact with the electrode member 310 may be formed on the second transfer body 222.

That is, the current supplied from the power supply part 300 moves to the first transfer body 221 through the first conductive surface 221a conducting electricity with the electrode member 310 on one side and then is supplied to the heating element 210, and the current passing through the heating element 210 moves to the second transfer member 222 and then moves back to the power supply part 300 through the second conductive surface 222a conducting electricity with the electrode member 310 on the other side.

In this case, as described above, the heat generated by the heating element 210 is transferred to the ceramic part 100 through the first transfer body 221 and the second transfer body 222. That is, the first transfer body 221 and the second transfer body 222 provide a path for the current supplied through the power supply part 300 to move to the heating element 210, and at the same time, they transfer heat generated in the heating element 210 to the ceramic part 100, and therefore, the first transfer body 221 and the second transfer body 222 are preferably formed of a material in which current and heat can move at the same time. For example, when the first transfer body 221 and the second transfer body 222 are formed of an aluminum material, the number of free electrons inside the aluminum is large such that the movement of current and the transfer of heat may be made smoothly. In addition, the present invention is not necessarily limited to such an aluminum material, and as long as it is an alloy material of aluminum and magnesium, or a material which is capable of transferring current and heat at the same time, such as gold, silver, tungsten and copper, various materials may be used to form the first transfer body 221 and the second transfer body 222.

A curved surface protruding toward the transfer member 220 may be formed on the outer peripheral surface of the above-described head 311.

That is, as the curved surface is formed on the head 311, the transfer member 220 and the head 311 come into electrical contact with each other in a point-contact manner, and noise generation due to friction may be prevented as the head 311 contacts the transfer member 220 in a point contact manner, and it is possible to effectively prevent the head 311 from being worn out. In addition, the head 311 may be formed in a coil shape, and through this, the head 311 may electrically contact each other while pressing the transfer member 220. The head 311 is not limited to the above-described shape, and may have any shape as long as it can electrically contact the transfer member 220.

As illustrated in FIG. 3, the head 311 supports the transfer member 220 in a manner of rotating together with the transfer member 220, and the body 312 supports the head 311 to rotate relatively. That is, the current supplied through the power supply part 300 moves to the heating part 200 while sequentially passing through the body 312 and the head 311. In addition, as the head 311 is configured to be rotatable, even when the transfer member 220 rotates, the head 311 rotates together to enable a stable current supply.

The electrical contact method between the transfer member 220 and the head 311 is not necessarily limited to a point contact manner, and a line contact method or a surface contact method may be sufficiently applied if noise generation or abrasion due to friction can be prevented.

As illustrated in FIG. 3, the body 312 may be provided with a spring 313 for providing a pressing force such that the head 311 presses the transfer member 220. This is to prevent an electrical contact from breaking while being spaced apart from each other as unintended abrasion occurs on the transfer member 220 or the head 311 during the long-term use of the thermotherapy device.

As illustrated in FIG. 2, an insulating member 230 may be provided between the first transfer body 221 and the second transfer body 222.

The current supplied through the power supply part 300 sequentially moves through the electrode member 310 on one side, the first transfer body 221, the heating element 210, the second transfer body 222 and the electrode member 31 on the other side, and if the first transfer body 221 and the second transfer body 222 are in direct contact with each other, a short circuit may occur and current supply through the power supply part 300 may not be made, and thus, direct electrical contact between the first transfer body 221 and the second transfer body 222 may be effectively prevented through the insulating member 230.

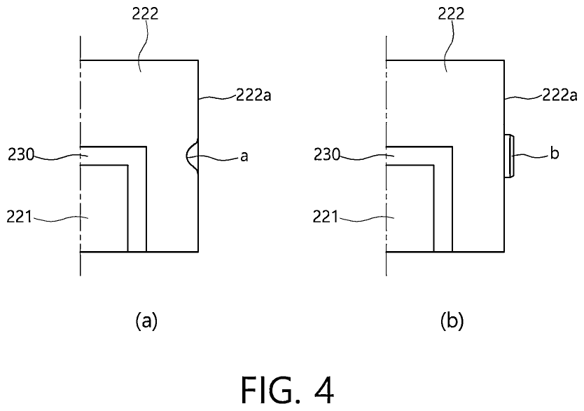

FIG. 4 is a cross-sectional view illustrating a heating part according to another exemplary embodiment of the present invention.

As illustrated in (a) of FIG. 4, the transfer member 220 may be formed with a conductive groove (a) which is in surface contact with the head 311 while surrounding a portion of the outer peripheral surface of the head 311. These conductive grooves (a) may be respectively formed on the first conductive surface 221a and the second conductive surface 222a, and since the head 311 is inserted into the conductive groove (a) and disposed to prevent the separation of the head 311, it enables stable electrical conduction.

Furthermore, since the conductive groove (a) and the head 311 are in surface contact with each other, when the transfer member 220 rotates in this state, the head 311 rotates together due to the frictional force formed due to the surface contact between the conductive groove (a) and the head 311. That is, since the transfer member 220 and the head 311 rotate together, abrasion does not occur therebetween, thereby effectively preventing the deterioration in durability of the transfer member 220. As the head 311 rotates, relative rotation occurs between the head 311 and the body 312, and as a result, when abrasion occurs, only the electrode member 310 needs to be replaced, and thus, maintenance and repair costs may be reduced.

Alternatively, as illustrated in (b) of FIG. 4, a protrusion-shaped conductive protrusion (b) may be formed. In this case, the head 311 of the electrode member 310 may be formed with a corresponding groove into which the conductive protrusion (b) is inserted, and since the conductive protrusion (b) is inserted into the corresponding groove, it is possible to effectively prevent the separation of the head 311.

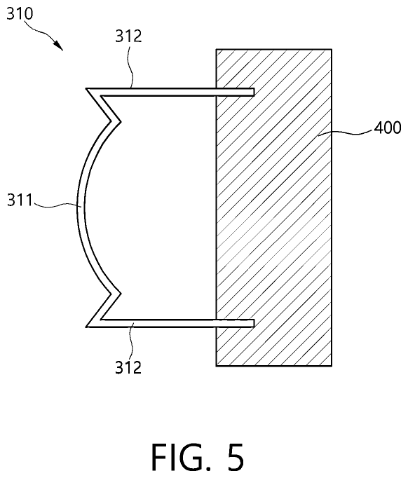

FIG. 5 is a cross-sectional view illustrating an electrode member according to another exemplary embodiment of the present invention.

As illustrated in FIG. 5, the head 311 may support the transfer member 220 to relatively rotate in a fixed state in a non-rotation state, and the body 312 may support the head 311. The head 311 and the body 312 may be manufactured simply by bending and deforming a plate-shaped plate.

In this case, the above-described head 311 may be formed with a deformable surface which is elastically deformed to press the transfer member 220. This deformable surface may be formed in a curved shape, and accordingly, the transfer member 220 and the head 311 are in electrical contact with each other in a point contact or line contact manner, and as the head 311 is in contact with the transfer member 220 in a point contact or line contact manner, it is possible to prevent noise generation due to friction, and it is possible to effectively prevent the abrasion of the head 311.

Furthermore, this deformable surface may be elastically deformed to press the transfer member 220, and if configured in this way, an unintentional abrasion phenomenon occurs in the transfer member 220 or the head 311 during the long-term use of the thermotherapy device, and accordingly, it is possible to prevent the electrical contact from breaking as they are spaced apart from each other.

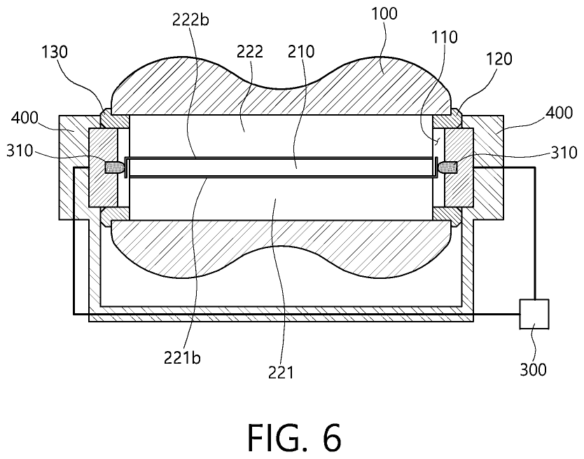

FIG. 6 is a cross-sectional view illustrating a state in which the ceramic part and the heating part are coupled according to still another exemplary embodiment of the present invention.

As illustrated in FIG. 6, the transfer member 220 may be provided with an electrode plate which is in electrical contact with the head 311. As illustrated in FIG. 6, the transfer member 220 may include a first transfer body 221 and a second transfer body 222 that are disposed to face each other, and the above-described heating element 210 may be provided between the first transfer body 221 and the second transfer body 222.

That is, by disposing the first transfer body 221 to contact one side surface of the heating element 210 and the second transfer body 222 to contact the other side surface of the heating element 210, the heat generated from the heating element 210 may be transferred to the first transfer body 221 and the second transfer body 222, and the heat transferred to the first transfer body 221 and the second transfer body 222 is transferred to the ceramic part 100. Transfer surfaces contacting the inner peripheral surface of the inner space 110 may be formed on the outer peripheral surface of the first transfer body 221 and the outer peripheral surface of the second transfer body 222, respectively.

In this way, if the heat generated through the heating element 210 is configured to be directly transferred to the ceramic part 100 in a conductive manner through the first transfer member 221 and the second transfer member 222, the heat transfer performance is improved such that the thermotherapy effect will be improved.

As described above, the transfer member 220 may be provided with an electrode plate which is in electrical contact with the head 311. That is, the first transfer body 221 may be provided with a first electrode plate 221b conducting electricity with the electrode member 310 on one side, and the second transfer body 222 may be provided with a second electrode plate 222b conducting electricity with the electrode member 310 on the other side, and a heating element 210 may be disposed between the first electrode plate 221b and the second electrode plate 222b that are disposed to face each other. In this case, a separate insulating member 230 may not be provided between the first electrode plate 221b and the second electrode plate 222b that are disposed to face each other.

In addition, bent surfaces conducting electricity with the electrode member 310 may be formed to be bent on the first electrode plate 221b and the second electrode plate 222, respectively, and the bent surface formed on the first electrode plate 221b needs to be spaced apart from the second electrode plate 222b so as not to conduct electricity with the second electrode plate 222b, and the bent surface formed on the second electrode plate 222b needs to be spaced apart from the first electrode plate 221b so as not to conduct electricity with the first electrode plate 221b.

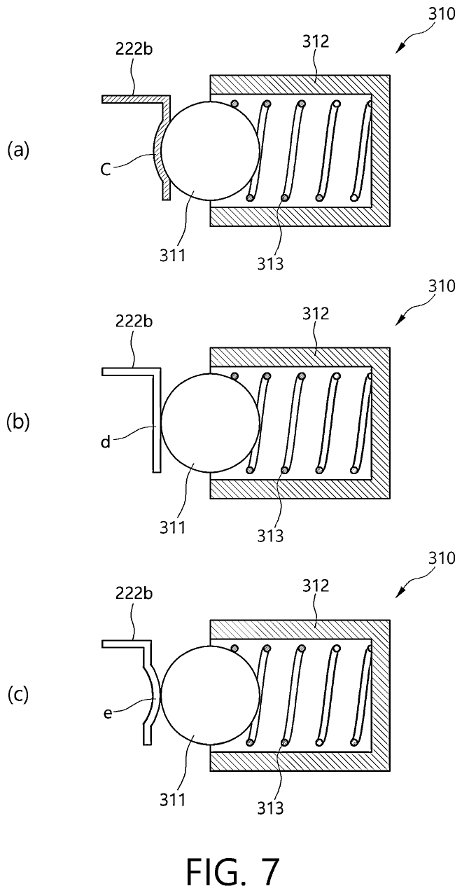

FIG. 7 is a cross-sectional view illustrating an electrode plate and an electrode member coupled to each other according to various exemplary embodiments of the present invention.

As illustrated in (a) of FIG. 7, a contact surface (c) that is in surface contact with the head 311 may be formed on the electrode plate described above while surrounding a portion of the outer peripheral surface of the head 311. The contact surface (c) may be formed on the first electrode plate 221b and the second electrode plate 222b, respectively, and since the head 311 is inserted into the contact surface (c), the head 311 is prevented from being separated so as to enable stable energization.

Furthermore, since the contact surface (c) and the head 311 are in surface contact with each other, when the transfer member 220 rotates in this state, the head 311 is rotated together by the frictional force formed due to the surface contact between the contact surface (c) and the head 311. That is, since the transfer member 220 and the head 311 rotate together, abrasion therebetween does not occur, and thus, it is possible to effectively prevent the deterioration in durability of the transfer member 220. As the head 311 rotates, relative rotation occurs between the head 311 and the body 312, and when abrasion occurs as a result, only the electrode member 310 needs to be replaced, and thus, maintenance and repair costs are reduced.

Alternatively, as illustrated in (b) of FIG. 7, a support surface (d) which is in point contact with the head 311 may be formed on the electrode plate described above. The support surface (d) has rigidity to support the head 311 without being deformed even when a pressing force is applied through the head 311, and through this, as the contact area between the head 311 and the support surface (d) is minimized, frictional resistance generated through the rotation of the transfer member 220 is minimized, thereby minimizing the deterioration in durability of the transfer member 220.

Moreover, as illustrated in (c) of FIG. 7, a protruding surface (e) which protrudes toward the head 311 may be formed on the above-described electrode plate. The protruding surface (e) may be elastically deformed to protrude toward the head 311, and through this, the protruding surface (e) presses the head 311. As described above, the body 312 of the electrode member 310 may be provided with a spring 313 for pressing the head 311, and when the protruding surface (e) of the electrode plate is elastically deformed to press the head 311, even if unintentional abrasion occurs on the transfer member 220 or the head 311 during the long-term use of the thermotherapy device, the electrode plate and the head 311 are not separated, and electrical contact may be made stably.

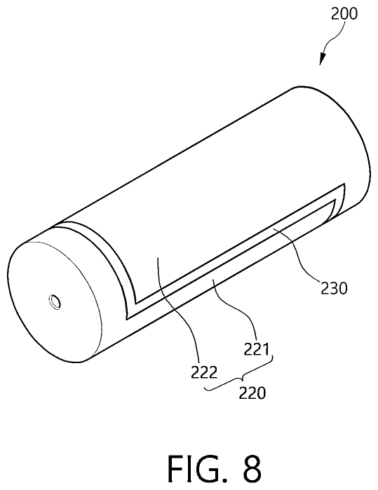

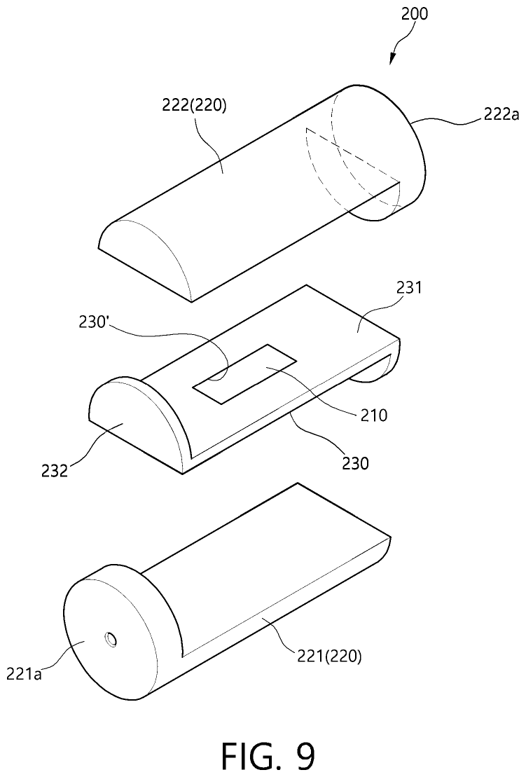

FIG. 8 is a perspective view illustrating a heating part according to an exemplary embodiment of the present invention, and FIG. 9 is an exploded perspective view of a heating part according to an exemplary embodiment of the present invention.

As illustrated in FIG. 8, an insulating member 230 is provided between the first transfer body 221 and the second transfer body 222, and as illustrated in FIG. 9, an insertion groove 230′ into which the heating element 210 is inserted may be formed on the insulating member 230.

As such, when the heating element 210 is inserted and fixed into the insertion groove 230′, not only the heating element 210 can be disposed in the correct position, but also the heating element 210 is disposed in the insulating member 230 in a modular manner, and thus, the assembly process of the heating part 200 may be simplified.

The insertion groove 230′ is formed to pass through one side surface and the other side surface of the insulating member 230, and while the heating element 210 is inserted into the insertion groove 230′, both side surfaces of the heating element 210 are exposed to contact the first transfer body 221 and the second transfer body 222. That is, in order for the both side surfaces of the heating element 210 to contact the first transfer body 221 and the second transfer body 222, it is important to configure the heating element 210 to be disposed in the center of the insertion groove 230′ without being biased to either side, based on a direction in which the heating element 210 is inserted into the insertion groove 230′.

To this end, the insertion groove 230′ may be provided with a separate stopper for fixing the position such that the heating element 210 can be disposed in the center of the insertion groove 230′.

As such, when the heating element 210 is disposed on the insulating member 230 in a modular manner, direct electrical contact between the first transfer member 221 and the second transfer member 222 is prevented, and the current which is supplied while sequentially passing through the electrode member 310 on one side and the first transfer body 221 moves to the heating element 210, and through this, heat is generated in the heating element 210, and afterwards, the current moves to the power supply part 300 while sequentially passing through the second transfer body 222 and the electrode member 310 on the other side.

In this case, as illustrated in FIG. 9, the first conductive surface 221a is formed to extend to surround the second transfer body 222, and the second conductive surface 222a is formed to extend to surround the first transfer body 221.

That is, on one side of the heating part 200 on which the electrode member 310 on one side is disposed, the first conductive surface 221a surrounds all of the second transfer body 222, and on the other side of the heating part 200 on which the electrode member 310 on the other side is disposed, the second conductive surface 222a surrounds all of the first transfer body 221, and thus, even if problems occur in which the heating part 200 is arbitrarily separated in the process of using the thermotherapy device, it is possible to stably prevent the electrode member 310 on one side from being energized with the second transfer body 222 or the electrode member 310 on the other side from being energized with the first transfer body 221.

Moreover, as described above, the first conductive surface 221a is disposed to surround the second transfer body 222, and the second conductive surface 222a is arranged to surround the first transfer body 221, but it is necessary to prevent electrical conduction between the first conductive surface 221a and the second transfer body 222 or between the second conductive surface 222a and the first transfer body 221.

To this end, the above-described insulating member 230 may be formed with abase surface 231 which is disposed between the first transfer body 221 and the second transfer body 222, and bent surfaces 232 which are disposed between the first conductive surface 221a and the second transfer body 222, and between the second conductive surface 222a and the first transfer body 221, respectively, and through this, it is possible to prevent electrical conduction between the first conductive surface 221a and the second transfer body 222 or between the second conductive surface 222a and the first transfer body 221.

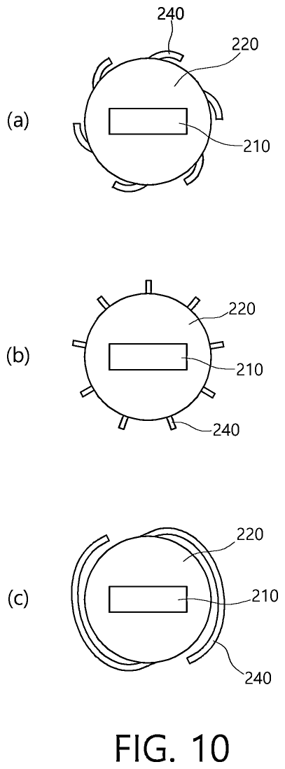

FIG. 10 is a side view illustrating a heating part according to still another exemplary embodiment of the present invention.

As illustrated in FIG. 10, the heating part 200 may be provided with an elastically deformable member 240 for pressing the inner peripheral surface of the conductive part 100. The elastically deformable member 240 is basically elastically deformed in the process of assembling the heating part 200 to the ceramic part 100, and it is elastically restored after the assembly to press the inner peripheral surface of the ceramic part 100. Therefore, since the heat generated by the heating part 200 can be directly transferred to the ceramic part 100 through the elastically deformable member 240 in a conductive manner, the thermotherapy effect is improved.

In this case, not only the heating part 200 but also the ceramic part 100 undergoes thermal expansion due to the heat generated through the heating part 200 in the process of using the thermotherapy device, and when the materials of the heating part 200 and the ceramic part 100 are different, the degree of thermal expansion is different. For example, when the ceramic part 100 is formed of a ceramic material and the heating part 200 is formed of an aluminum material, the degree of thermal expansion of the heating part 200 is greater than the degree of thermal expansion of the ceramic part 100, and as a result, in the process of using the thermotherapy device, phenomena in which the heating part 200 presses the inner peripheral surface of the ceramic part 100 may occur, and as a result, there may be problems in that the ceramic part 100 is damaged. Therefore, as described above, when the elastically deformable member 240 is provided in the heating part 200, the force pressing the inner peripheral surface of the ceramic part 100 is reduced as the elastically deformable member 240 is elastically deformed when the heating part 200 thermally expands, and thus, it is possible to effectively prevent the ceramic part 100 from being damaged.

At least one of these elastically deformable members 240 may be provided along the periphery of the heating part 200, and as illustrated in (a) of FIG. 10, it is preferable that the front end of the elastically deformable member 240 is disposed adjacent to the outer peripheral surface of the heating part 200 and configured to be spaced apart from the outer peripheral surface of the heating part 200 by a predetermined distance so as to be elastically deformable. When configured in this way, if thermal expansion occurs in the heating part 200, the elastically deformable member 240 is elastically deformed such that the separation distance between the front end of the elastically deformable member 240 and the outer peripheral surface of the heating part 200 decreases, and the force pressing the inner peripheral surface of the ceramic part 100 is reduced. Alternatively, as illustrated in (b) of FIG. 10, it is also possible to configure the elastically deformable member 240 to extend in the radial direction. When configured in this way, if thermal expansion occurs in the heating part 200, the elastically deformable member 240 is elastically deformed in a bending manner, and the force pressing the inner peripheral surface of the ceramic part 100 is reduced. Moreover, as illustrated in (c) of FIG. 10, it is also possible to configure the elastically deformable member 240 to partially cover the outer peripheral surface of the heating part 200. That is, for the basic operation in which the elastically deformable member 240 is elastically deformed during the thermal expansion of the heating part 200, (a) and (c) of FIG. 10 are similar, but the elastically deformable member 240 illustrated in (c) of FIG. 10 is formed to be longer than the elastically deformable member 240 illustrated in (a) of FIG. 10. When configured in this way, a contact area between the elastically deformable member 240 and the inner peripheral surface of the ceramic part 100 is increased such that the heat transfer effect can be improved.

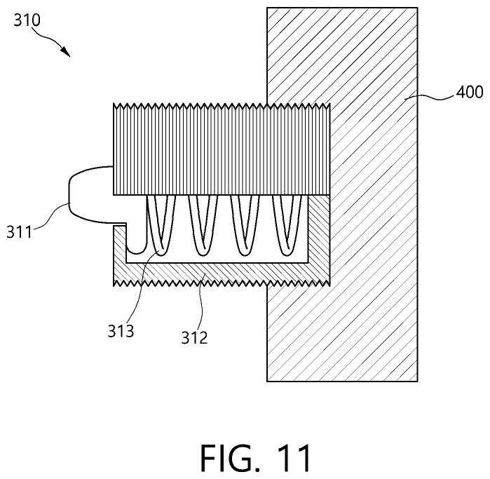

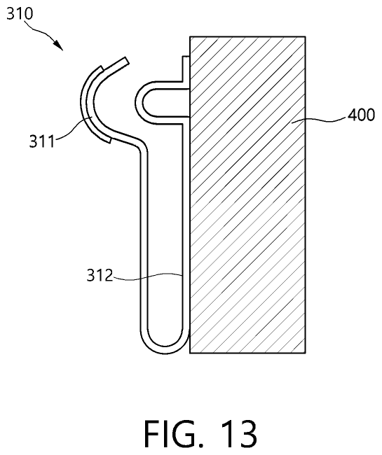

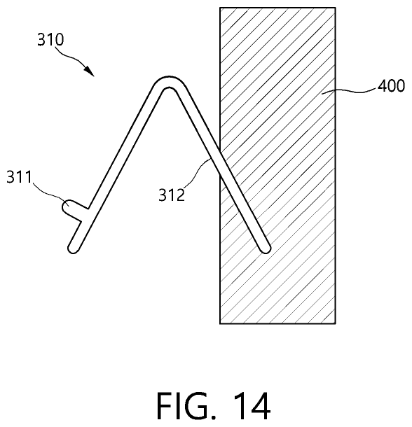

FIGS. 11 to 14 are cross-sectional views illustrating an electrode member according to various exemplary embodiments of the present invention.

As illustrated in FIG. 11, the head 311 of the electrode member 310 may be provided with ribs which are formed to extend outwardly in the radial direction, and the body 312 may be provided with corresponding ribs on which these ribs are caught, and through this, it is possible to effectively prevent the head 311 from being completely separated from the body 312.

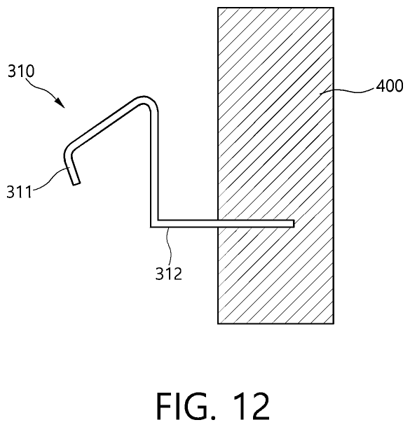

As illustrated in FIGS. 12 to 14, the electrode member 310 may use an electrode member 310 in the form of a leaf spring, and the electrode member 310 may be provided with a body 312 which is fixedly disposed on the support part 400, and a head 311 which is formed to be integrally extended from the body 312 and elastically deformed to apply an elastic force toward the heating part 200.

Although an exemplary embodiment of the present invention has been described above, the spirit of the present invention is not limited to the exemplary embodiments presented herein, and a person skilled in the art who understands the spirit of the present invention may easily suggest other exemplary embodiments by modifying, changing, deleting or adding components within the scope of the same spirit, but it can be said that this will also fall within the spirit of the present invention.

1. A thermotherapy device, comprising:
a ceramic part having an inner space formed therein;
a heating part which is inserted in the inner space and has a heating element for generating heat so as to heat the ceramic part, and a transfer member for transferring the heat generated by the heating element to the ceramic part;
a power supply part having an electrode member so as to supply an electric current to the heating part; and
a support part for supporting the ceramic part,
wherein the heating part rotates relative to the electrode member such that the heating part rotates together with the ceramic part.

2. The thermotherapy device of claim 1, wherein the electrode member comprises a head which electrically contacts the transfer member and supports the transfer member so as to be rotatable, and a body which is fixed to the support part to support the head.

3. The thermotherapy device of claim 2, wherein the head supports the transfer member in a manner of rotating with the transfer member, and
wherein the body supports the head to rotate relatively.

4. The thermotherapy device of claim 3, wherein the body is provided with a spring for providing a pressing force such that the head presses the transfer member.

5. The thermotherapy device of claim 3, wherein a conducting groove is formed in the transfer member to be in surface contact with the head while surrounding a portion of the outer peripheral surface of the head.

6. The thermotherapy device of claim 2, wherein the head supports the transfer member to relatively rotate in a fixed state in a non-rotating state, and
wherein the body supports the head.

7. The thermotherapy device of claim 6, wherein a deformable surface which is elastically deformed to press the transfer member is formed on the head.

8. The thermotherapy device of claim 7, wherein the deformable surface is elastically deformed in such a way that it protrudes toward the transfer member.

9. The thermotherapy device of claim 2, wherein the transfer member is provided with an electrode plate that electrically contacts the head.

10. The thermotherapy device of claim 9, wherein a contact surface is formed on the electrode plate to be in surface contact with the head while surrounding a portion of the outer peripheral surface of the head.

11. The thermotherapy device of claim 1, wherein the heating part is provided with an elastically deformable member that presses the inner peripheral surface of the ceramic part.
